(19) United States
(12) Patent Application Publication
(10) Pub. No.: US 2008/0097377 A1
(43) Pub. Date: Apr. 24, 2008

(54) Temperature-controlled catheter system and method

(75) Inventors: Eric T. Crumpler, Miami, FL (US); Mahwish Ahmed, Pembroke Pines, FL (US); Mayssaa El-Churafa, Pembroke Pines, FL (US); Haroldo S. Silva, Sunny Isles Beach, FL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE FLORIDA INTERNATIONAL UNIVERSITY BOARD OF TRUSTEES, Miami, FL (US)

(21) Appl. No.: 11/451,894

(22) Filed: Jun. 13, 2006

(51) Int. Cl. A61M 25/00

(52) U.S. Cl. 604/506; 604/113

(57) ABSTRACT

A catheter system and method are provided for delivering a substance, such as medication, to body tissue. The catheter system includes a medication delivery tube and at least one heat transfer fluid passage that at least partially surrounds the medication delivery tube. The catheter system may be used to deliver temperature-sensitive substances, such as, for example thermo-sensitive polymers, to living tissue.

### FIELD OF THE INVENTION

The invention is related generally to catheters, and specifically to catheters for delivery of substances at controlled temperatures.

### BACKGROUND

Catheters are widely used in the medical field, for various procedures such as, for example, angioplasty, stent delivery, cryogenic, and drug delivery. Catheters for local drug delivery have been developed to offer regional or site-specific delivery. Site-specific catheters permit an improved delivery of a therapeutic agent to be infused at specific sites of living tissue. By using passive or active diffusion properties, catheters are able to deliver pharmaceuticals, including temperature-sensitive drugs, as well as genetic agents, to living tissues.

Stimuli sensitive polymers, such as hydrogels and liposome drug carriers, respond to changes in temperature and metabolites. Thermo-sensitive polymers are water soluble below a lower critical solution temperature and are water insoluble above the lower critical solution temperature. For clinical applications, these polymers may be formulated to become insoluble (e.g., form a gel), at body temperature. Promising applications of such polymers include cellular therapy, embolization procedures, tissue sealants, and for targeted delivery of medical isotopes or chemotherapy drugs to treat problematic solid tumors, such as those associated with liver, pancreas, brain, breast, and prostate tissue.

However, catheters that are currently commercially available lack an efficient temperature-controlled delivery system for such polymer-type materials.

The present invention is directed to overcoming one or more of the problems or disadvantages associated with the prior art.

### SUMMARY

In accordance with one aspect of the invention, a catheter system includes a medication delivery tube and at least one heat transfer fluid passage that at least partially surrounds the medication delivery tube. The medication delivery tube may terminate in a needle portion. The catheter system may further include an insulating portion that at least partially surrounds the heat transfer fluid passage. The heat transfer fluid passage and the medication delivery tube may be disposed in a coaxial configuration. The heat transfer fluid passage may include an inflow passage and an outflow passage, one or both of which may be configured, for example, as a C-shaped lumen passage. The heat transfer fluid passage may include a tube coiled around the medication delivery tube.

In accordance with another aspect of the invention, a catheter system includes a medication delivery tube and at least one heat transfer fluid passage that is disposed adjacent to the medication delivery tube. An insulating portion may be provided that at least partially surrounds the heat transfer fluid passage, and the heat transfer fluid passage and the medication delivery tube may be disposed in a coaxial configuration. The heat transfer fluid passage may include an inflow passage and an outflow passage, one or both of which may be configured as a C-shaped lumen passage.

In accordance with yet another aspect of the invention, a method of delivering a substance into living tissue is provided. The method includes providing a catheter that includes a delivery tube and a heat transfer fluid passage that at least partially surrounds the delivery tube, maintaining a desired delivery temperature by circulating a heat transfer fluid through the heat transfer fluid passage, and delivering the substance into living tissue through the delivery tube at the desired delivery temperature. The heat transfer fluid may be, for example, water, or, alternatively, saline solution. The substance may be delivered in liquid form and may be in gel form at a temperature approximately equal to a temperature of the living tissue.

### BRIEF DESCRIPTION OF THE DRAWINGS

FIG. 1 is a partially cross-sectional diagrammatic perspective view of a first embodiment of a catheter system;

FIG. 2 is a partially cross-sectional diagrammatic perspective view of a second embodiment of a catheter system; and

FIG. 3 is a partially cross-sectional diagrammatic perspective view of a third embodiment of a catheter system.

### DETAILED DESCRIPTION

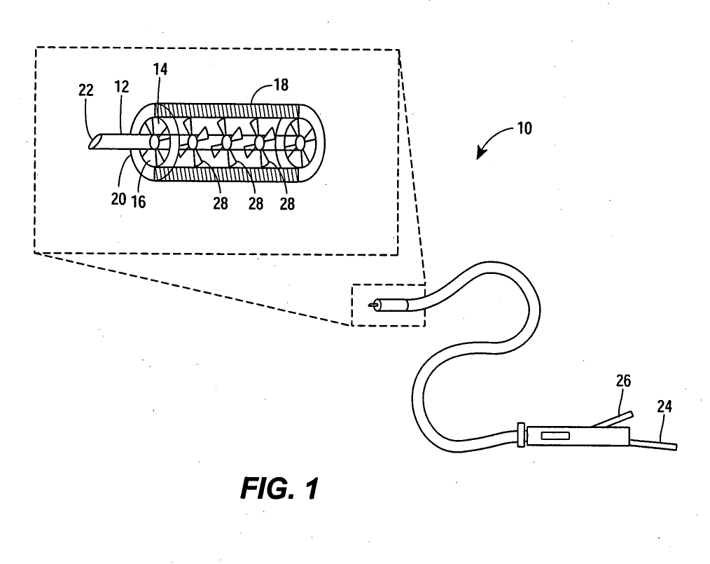

A first embodiment of a catheter system in accordance with one aspect of the invention is depicted in FIG. 1, in which as catheter system 10 includes a medication delivery tube 12 that is at least partially surrounded in a coaxial fashion by a heat transfer fluid passage 14. The heat transfer fluid passage 14 may be at least partially surrounded by an insulating membrane 16 that is in turn housed within an outer sheath 18. A distal end 20 of the catheter system 10 may be unsealed, and thus heat transfer fluid may exit the catheter system 10 at the distal end 20. The medication delivery tube 12 may terminate in a needle portion 22.

The medication delivery tube 12 may be in fluid communication with an injection port 24 that may be, for example, connected to a syringe (not shown) for drug delivery purposes. The heat transfer fluid passage 14 may be in fluid communication with an inlet port 26 that may be used to continuously flow a heat transfer fluid, such as, for example, chilled water, within the heat transfer fluid passage 14, using an external pump (not shown). A plurality of spacer elements 28 may be provided to securely position the medication delivery tube 12 with respect to the insulating membrane 16.

A substance, such as a medication (not shown) may thus be injected through the injection port 24, while heat transfer fluid is flowing through the heat transfer fluid passage 14, thereby maintaining the medication at a regulated desired temperature (e.g., cooled below body temperature, for example, below about 98.6 degrees Fahrenheit). Other fluids, such as, for example, saline solution, may be used for the heat transfer fluid.

The insulating membrane 16 may be made from polyvinyl chloride (PVC), or any other suitable material, such as polytetrafluoroethylene (PTFE) or polyimide. The outer sheath 18, the medication delivery tube 12, and the spacer elements 28 may also be composed of PVC, PTFE, or Polyimide material, and the needle portion 22 may be made of stainless steel hypotubing.

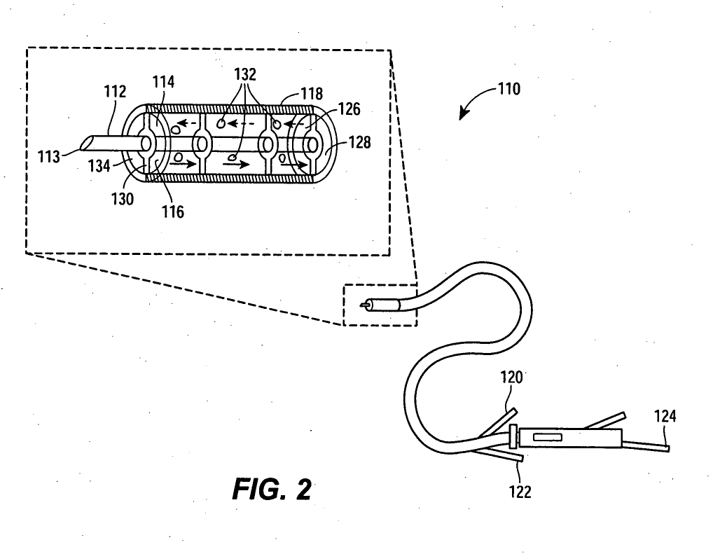

A second embodiment of the invention is depicted in FIG. 2, in which a catheter system 116 includes a medication delivery tube 112 that is at least partially surrounded by a heat transfer fluid passage 114. An insulating membrane 116 at least partially surrounds the heat transfer fluid passage 114, and that is in turn housed within an outer sheath 118. The medication delivery tube 112 may be in fluid communication with an injection port 124. The heat transfer fluid passage 114 may be in fluid communication with an inlet port 120 and an outlet port 122. The medication delivery tube 112 may terminate in a needle portion 113.

The heat transfer fluid passage 114 may be divided by a wall 130 into an inflow passage 126 and an outflow passage 128. The wall 130 may include a plurality of openings 132 to facilitate flow of heat transfer fluid between the inflow passage 124 and the outflow passage 126. The inflow passage 126 and the outflow passage 128 may be configured as C-shaped lumen passages. The heat transfer fluid passage 114 may be sealed at a distal end 134.

The insulating membrane 116 may be made from polyvinyl chloride (PVC), or any other suitable material, such as polytetrafluoroethylene (PTFE) or polyimide. The outer sheath 118 and the medication delivery tube 112 may also be composed of PVC, PTFE, or Polyimide material, and the needle portion 113 may be made of stainless steel hypotubing.

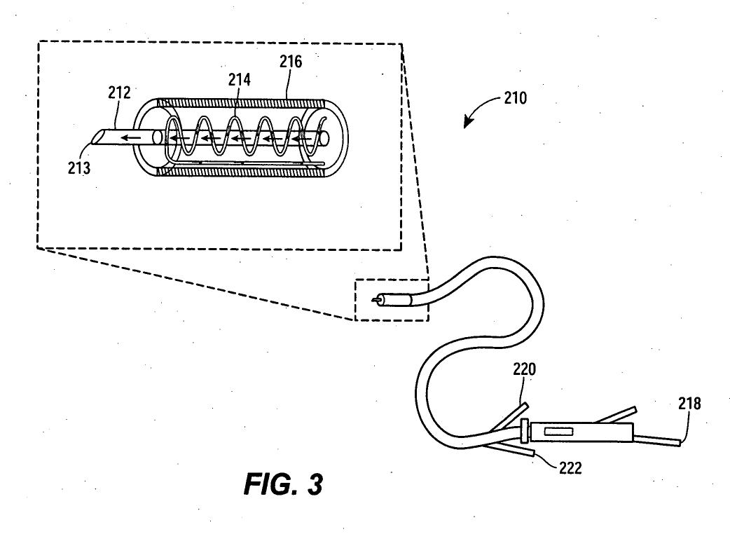

A third embodiment of the invention is depicted in FIG. 3, in which a catheter system 210 includes a medication delivery tube 212 that is partially surrounded by a heat transfer fluid passage 214, that may be, for example coiled around the medication delivery tube 212, or otherwise placed in close proximity or adjacent to the medication delivery tube 212. The medication delivery tube 212 may terminate in a needle portion 213.

An insulating membrane 216 may at least partially surround the heat transfer fluid passage 214 and the medication delivery tube 212. The medication delivery tube 212 may be connected in fluid communication with an injection port 218, and the heat transfer fluid passage 214 may be in fluid communication with an inlet port 220 and an outlet port 222.

The insulating membrane 216 may be made from polyvinyl chloride (PVC), or any other suitable material, such as polytetrafluoroethylene (PTFE) or polyimide. The medication delivery tube 212 may also be composed of PVC, PTFE, or Polyimide material, and the needle portion 213 may be made of stainless steel hypotubing.

Of course, it should be understood that a range of changes and modifications can be made to the preferred embodiments described above. It is therefore intended that the foregoing detailed description be regarded as illustrative rather than limiting and that it be understood that it is the following claims, including all equivalents, which are intended to define the scope of this invention.

What is claimed is:

1. A catheter system comprising:
a medication delivery tube; and
at least one heat transfer fluid passage that at least partially surrounds the medication delivery tube.

2. The catheter system of claim 1, further including an insulating portion that at least partially surrounds the heat transfer fluid passage.

3. The catheter system of claim 1, wherein the heat transfer fluid passage and the medication delivery tube are disposed in a coaxial configuration.

4. The catheter system of claim 1, where in the heat transfer fluid passage includes an inflow passage and an outflow passage.

5. The catheter system of claim 4, wherein the inflow passage and the outflow passage are each configured as C-shaped lumen passages.

6. The catheter system of claim 1, wherein the heat transfer fluid passage includes a tube coiled around the medication delivery tube.

7. The catheter system of claim 1, wherein the medication delivery tube terminates in a needle portion.

8. A catheter system comprising:
a medication delivery tube; and
at least one heat transfer fluid passage that is disposed adjacent to the medication delivery tube.

9. The catheter system of claim 8, further including an insulating portion that at least partially surrounds the heat transfer fluid passage.

10. The catheter system of claim 8, wherein the heat transfer fluid passage and the medication delivery tube are disposed in a coaxial configuration.

11. The catheter system of claim 8, where in the heat transfer fluid passage includes an inflow passage and an outflow passage.

12. The catheter system of claim 11, wherein at least one of the inflow passage and the outflow passage is configured as a C -shaped lumen passage.

13. The catheter system of claim 8, wherein the heat transfer fluid passage includes a tube coiled around the medication delivery tube.

14. The catheter system of claim 8, wherein the medication delivery tube terminates in a needle portion.

15. A method of delivering a substance into living tissue comprising:
providing a catheter that includes a delivery tube and a heat transfer fluid passage that at least partially surrounds the delivery tube;
maintaining a desired delivery temperature by circulating a heat transfer fluid through the heat transfer fluid passage; and
delivering the substance into living tissue through the delivery tube at the desired delivery temperature.

16. The method of claim 15, wherein the heat transfer fluid is water.

17. The method of claim 15, wherein the heat transfer fluid is chilled to a temperature below about 98.6 degrees Fahrenheit.

18. The method of claim 15, wherein the substance is delivered in liquid form, and wherein the substance is in gel form at a temperature approximately equal to a temperature of the living tissue.

19. The method of claim 15, wherein the heat transfer fluid is saline solution.
